(19) United States
(12) Patent Application Publication
(10) Pub. No.: US 2005/0209510 A1
(43) Pub. Date: Sep. 22, 2005

(54) Wound retractor device

(76) Inventors: Frank Bonadio, Bray (IE); Trevor Vaugh, Birr (IE); John Butler, Blackrock (IE); Catherine Deegan, Clontarf (IE)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/086,659

(22) Filed: Mar. 23, 2005

Related U.S. Application Data
(63) Continuation-in-part of application No. 10/374,523, filed on Feb. 27, 2003.
Application No. 10/374,523 is a continuation of application No. 09/849,341, filed on May 7, 2001, now Pat. No. 6,582,364.
Application No. 09/849,341 is a continuation of application No. 09/688,138, filed on Oct. 16, 2000, now Pat. No. 6,254,534.
(60) Provisional application No. 60/555,408, filed on Mar. 23, 2004.

(30) Foreign Application Priority Data
Feb. 18, 2000 (EP) 00650010.2
Oct. 14, 1999 (IE) 990861
Dec. 16, 1999 (IE) 991053

(51) Int. Cl. A61B 1/32

(52) U.S. Cl. 600/208

(57) ABSTRACT

A wound retractor device (1) comprises a distal ring member (2) for insertion into a wound opening (3), a proximal ring member (4) for location externally of the wound opening (3), and a cylindrical connecting sleeve (5), which extends between the distal ring member (2) and the proximal ring member (4), to retract laterally the sides of the wound opening (3). The device (1) further comprises a rigid ring-shaped platform member (6) to support one or more medical devices in a desired location and/or orientation. The platform member (6) is suitable for location, in use, externally of the wound opening (3). The device (1) also comprises a medical device (16) supported on the platform member (6). The medical device (16) comprises a body portion (9), a gripping member (11), and a rigid arm member (10) to connect the gripping member (11) to the body portion (9). A medical instrument (14) may be then positioned in a “C”-shaped opening (21) of the gripping member (11). The location and/or orientation of the medical instrument (14) may be adjusted by adjusting the arm member (10) and/or the gripping member (11).

This is a continuation-in-part of application Ser. No. 10/374,523, filed Feb. 27, 2003, which is a continuation of application Ser. No. 09/849,341, filed May 7, 2001, now U.S. Pat. No. 6,582,364, which is a continuation of application Ser. No. 09/688,138, filed Oct. 16, 2000, now U.S. Pat. No. 6,254,534 B1. This application claims the benefit of U.S. Provisional Application No. 60/555,408, filed on Mar. 23, 2004. The contents of all of the above-listed applications are incorporated herein by reference.

### INTRODUCTION

This invention relates to a wound retractor device, and to a method for retracting a wound opening.

### STATEMENTS OF INVENTION

According to the invention there is provided a wound retractor device comprising:
a distal member for insertion into a wound opening;
a proximal member for location externally of the wound opening;
a connecting member extending between the distal member and the proximal member to retract laterally the sides of the wound opening; and
a support to support one or more medical devices in a desired location and/or orientation.

In one embodiment the proximal member comprises the support.

In another embodiment the support comprises at least one platform member for location externally of a wound opening. The platform member may be mountable to the proximal member. The platform member may be mountable to the proximal member in a snap-fit manner. The platform member may comprise a female recess into which the proximal member is receivable to mount the platform member to the proximal member. In one case the female recess is substantially “C”-shaped in cross-section. In one case an opening of the female recess faces radially inwardly and/or faces radially outwardly.

The proximal member may comprise a ring. The platform member may comprise a ring.

In one embodiment the device comprises a medical device configured to be supported by the support. The medical device may be mountable to the support. The medical device may be mountable to the support in a snap-fit manner. In one case a body portion of the medical device comprises a female recess into which the support is receivable to mount the medical device to the support. The female recess may be substantially “C”-shaped in cross-section. An opening of the female recess may face radially inwardly, and/or face radially outwardly.

In one case the body portion of the medical device extends around a relatively large part of the circumference of the support. The body portion of the medical device may extend around more than 50% of the circumference of the support.

In another case the body portion of the medical device extends around a relatively small part of the circumference of the support. The body portion of the medical device may extend around less than 10% of the circumference of the support.

In another embodiment the medical device comprises a proximal anchor member for anchoring the medical device to the support to mount the medical device to the support. The proximal anchor member may be configured to be looped around a proximal side of the support and anchored at a distal side of the support. In one case the proximal anchor member comprises a ring.

The medical device may comprise a holder to hold a medical instrument in a desired location and/or orientation. The holder may comprise a gripping member which is substantially “C”-shaped in cross-section. In one case the holder is adjustable to adjust the location and/or orientation of the medical instrument. The holder may be connected to a body portion of the medical device by means of an arm member. The holder may be pivotally mounted to the arm member. In one case the arm member is pivotally mounted to the body portion of the medical device. The arm member may be extendable.

The arm member may be rigid. The arm member may be flexible. In one case the arm member comprises a wire, or cable, or cord.

In another embodiment the holder comprises a tray.

In one case the medical device comprises artificial vision means to view a wound interior. The artificial vision means may comprise a scope for insertion through a wound opening. The location and/or orientation of the scope may be adjustable. In one case the artificial vision means comprises a monitor to which the scope is operably connected. The location and/or orientation of the monitor may be adjustable.

In another embodiment the medical device comprises a retainer to retain internal organs in a desired configuration. The retainer may comprise a sleeve member configured to be extended through a wound opening into a wound interior. In one case the retainer comprises a maintainer to maintain a distal opening of the sleeve member in an open configuration. The maintainer may comprise a distal ring member at a distal end of the sleeve member. The maintainer may comprise an expandable chamber at a distal end of the sleeve member.

In another case the retainer comprises an outer envelope of pliable material, the envelope having an evacuation port, and at least two sheets of material arranged in face to face relation in the envelope, the retainer having a malleable forming configuration in which the sheets are relatively free to slide over one another in manipulating the retainer to a desired shape, and a form retained configuration in which the sheets are restrained from sliding relative to one another on evacuation of the envelope.

In another aspect the invention provides a method for retracting a wound opening, the method comprising the steps of:

providing a wound retractor device comprising a distal member, a proximal member, and a connecting member extending between the distal member and the proximal member;

inserting the distal member into the wound opening, and locating the proximal member externally of the wound opening, with the connecting member extending between the distal member and the proximal member to retract laterally the sides of the wound opening; and

supporting one or more medical devices on the wound retractor device in a desired location and/or orientation.

In one embodiment the medical device is supported on the proximal member.

In another embodiment the wound retractor device comprises at least one platform member located externally of the wound opening, and the medical device is supported on the platform member. The method may comprise the step of mounting the platform member to the proximal member. In one case the method comprises the step of mounting a medical device to the wound retractor device. The method may comprise the step of anchoring the medical device to the wound retractor device. In one case the method comprises the step of using the medical device to hold a medical instrument in a desired location and/or orientation.

The method may comprise the step of using the medical device to view a wound interior.

The method may comprise the step of using the medical device to retain internal organs in a desired configuration.

### BRIEF DESCRIPTION OF THE DRAWINGS

The invention will be more clearly understood from the following description of some embodiments thereof, given by way of example only, with reference to the accompanying drawings, in which:

FIGS. 1 and 2 are plan views of a wound retractor device according to the invention;

FIGS. 3 to 11 are partially cross-sectional, side views of the device of FIGS. 1 and 2, in use;

FIG. 12 is a partially cross-sectional, side view of a part of the device of FIGS. 1 and 2, in use;

FIG. 13 is a plan view of another wound retractor device according to the invention;

FIGS. 14 to 17 are partially cross-sectional, side views of a further wound retractor device according to the invention, in use;

FIG. 18 is a partially cross-sectional, side view of another wound retractor device according to the invention, in use;

FIG. 19 is a plan view of another wound retractor device according to the invention;

FIG. 20 is a partially cross-sectional, side view of the device of FIG. 19, in use;

FIG. 21 is a plan view of a further wound retractor device according to the invention;

FIG. 22 is a cross-sectional, side view of the device of FIG. 21, in use;

FIG. 22(a) is a cross-sectional, side view of another wound retractor device according to the invention, in use;

FIG. 23 is a cross-sectional, side view of a part of another wound retractor device according to the invention;

FIG. 24 is a cross-sectional, side view of the device of FIG. 23, in use;

FIG. 25 is a partially cross-sectional, side view of another wound retractor device according to the invention, in use;

FIG. 26 is a perspective view of a part of a further wound retractor device according to the invention;

FIGS. 27 and 28 are cross-sectional, side views of the device of FIG. 26, in use;

FIG. 29 is a partially cross-sectional, side view of another wound retractor device according to the invention; and

FIG. 30 is a cross-sectional, side view of a further wound retractor device according to the invention.

### DETAILED DESCRIPTION

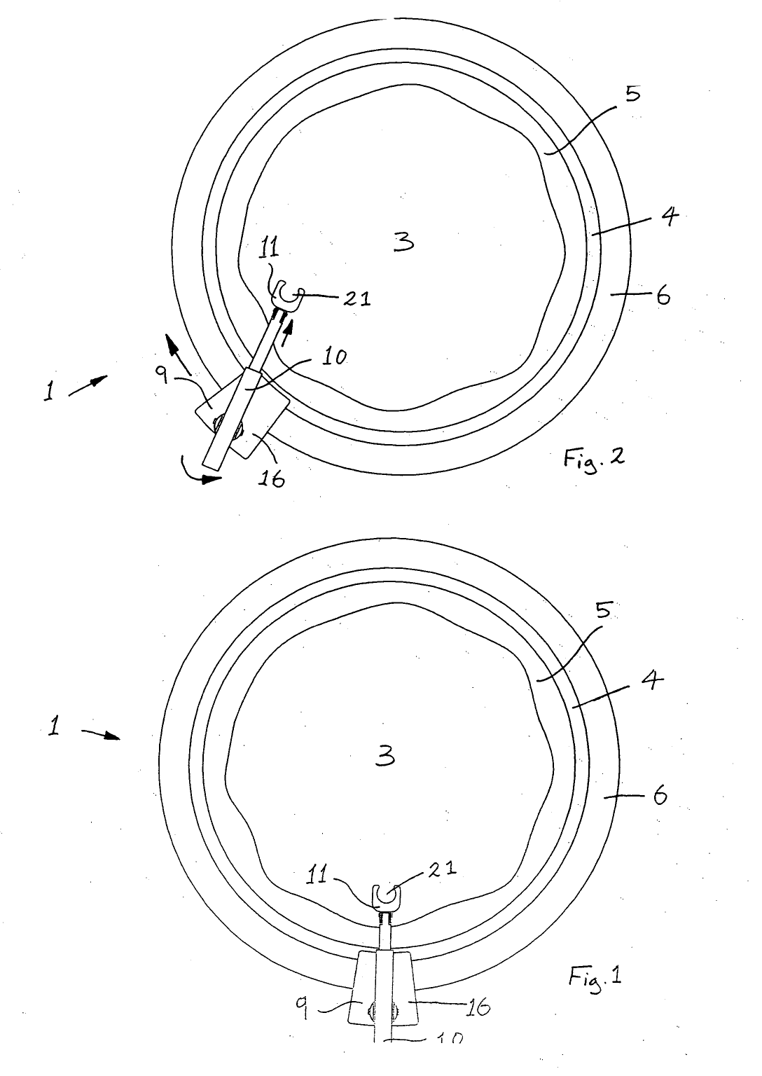

Referring to the drawings, and initially to FIGS. 1 to 11 thereof, there is illustrated a wound retractor device 1 according to the invention.

The device 1 comprises a distal ring member 2 for insertion into a wound opening 3, a proximal ring member 4 for location externally of the wound opening 3, and a connecting member. In this case, the connecting member is provided in the form of a cylindrical connecting sleeve 5, which extends between the distal ring member 2 and the proximal ring member 4, to retract laterally the sides of the wound opening 3.

As illustrated in FIG. 3, the sleeve 5 is fixedly attached to the proximal ring member 4 at a first end of the sleeve 5, extends distally to the distal ring member 2 in a first layer. The sleeve 5 is looped around the distal ring member 2 and extends proximally to the proximal ring member 4 in a second layer. The second layer is located radially outwardly of the first layer. A second end of the sleeve 5 remains detached from the proximal ring member 4. The arrangement of the distal ring member 2, proximal ring member 4 and connecting sleeve 5 provides a self-locking retractor. This arrangement is described in further detail in International patent application No. PCT/IE2003/000141, the relevant contents of which are incorporated herein by reference.

The device 1 further comprises a support to support one or more medical devices in a desired location and/or orientation. In this case, the support is provided by a rigid, ring-shaped platform member 6 suitable for location, in use, externally of the wound opening 3. The platform member 6 is substantially “C”-shaped in cross-section, and comprises a female recess 7 into which the proximal ring member 4 is receivable to mount the platform member 6 to the proximal ring member 4 in a snap-fit manner.

As illustrated in FIG. 3, the opening 8 of the female recess 7 faces radially inwardly.

The device 1 also comprises a medical device 16 supported on the platform member 6. The medical device 16 comprises a body portion 9, a gripping member 11, and a rigid arm member 10 to connect the gripping member 11 to the body portion 9.

The body portion 9 is substantially “C”-shaped in cross-section, and comprises a female recess 12 into which the platform member 6 is receivable to mount the body portion 9 to the platform member 6 in a snap-fit manner. As illustrated in FIG. 3, the opening 13 of the female recess 12 faces radially inwardly.

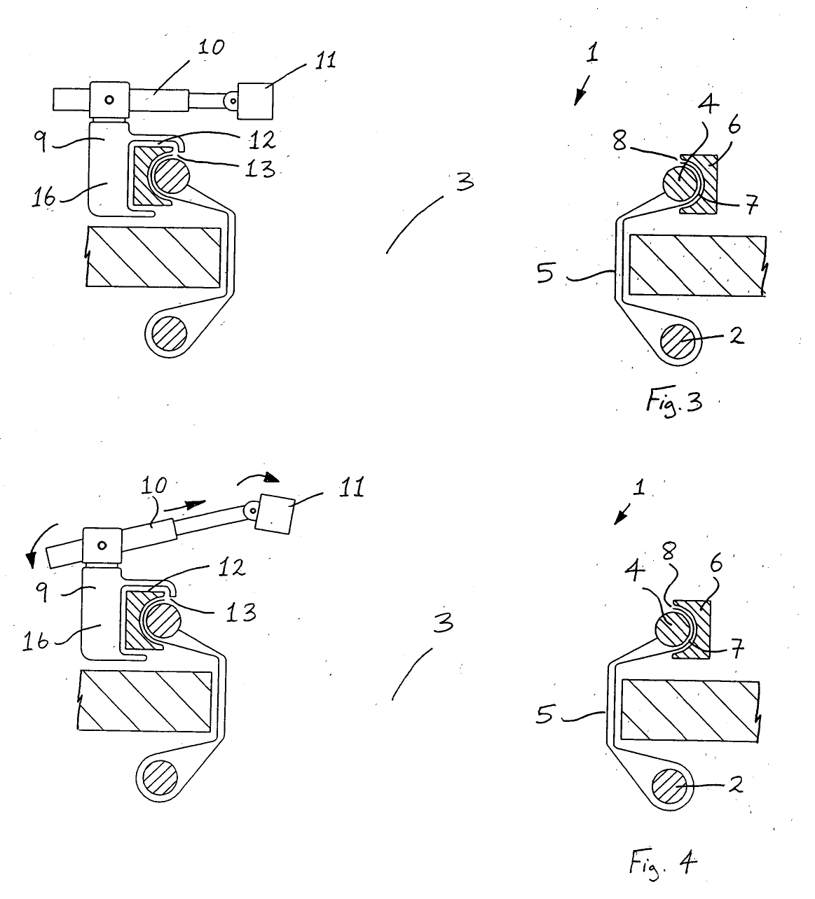

As illustrated in FIGS. 1 and 2, the body portion 9 extends around a relatively small part of the circumference of the platform member 6. In this case, the body portion 9 extends around less than 10% of the circumference of the platform member 6.

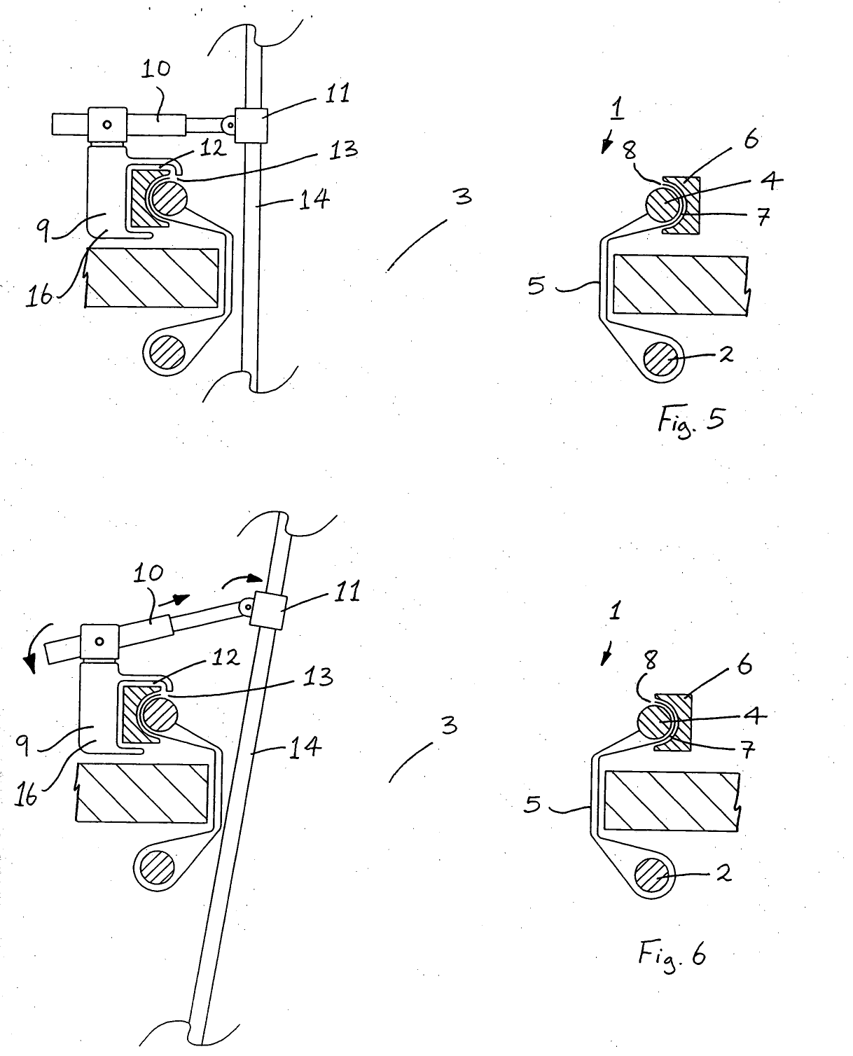

The gripping member 11 is substantially “C”-shaped in cross-section, as illustrated in FIGS. 1 and 2, and is configured to hold a medical instrument 14 in a desired location and/or orientation, as illustrated in FIGS. 5 and 6.

The arm member 10 is pivotally mounted to the body portion 9 to facilitate pivoting of the arm member 10 relative to the body portion 9 in the horizontal plane (FIG. 2), and also in the vertical plane (FIG. 4). In addition, the gripping member 11 is pivotally mounted to the arm member 10 to facilitate pivoting of the gripping member 11 relative to the arm member 10 in the vertical plane (FIG. 4). Also the body portion 9 may slide along the platform member 6, while the body portion 9 is mounted to the platform member 6 (FIG. 2). Furthermore, the arm member 10 is telescopically extendable (FIG. 4). In this manner, the gripping member 11 may be adjusted in the three dimensions, as illustrated in FIGS. 3 and 4, to adjust the location and/or orientation of the medical instrument 14 as desired, as illustrated in FIGS. 5 and 6.

In use, the distal ring member 2 is inserted into the wound opening 3, and the proximal ring member 4 and is located externally of the wound opening 3, with the connecting sleeve 5 extending between the distal ring member 2 and the proximal ring member 4. The sleeve 5 is then pulled proximally to retract laterally the sides of the wound opening 3. Retraction of the wound opening 3 in this manner is described in further detail in International patent application No. PCT/IE2003/000141, the relevant contents of which are incorporated herein by reference.

The platform member 6 may then be mounted to the proximal ring member 4 in a snap-fit manner. The body portion 9 is mounted to the platform member 6 in a snap-fit manner (FIG. 3). In this manner, the medical device 16 is supported on the platform member 6. The location and/or orientation of the gripping member 11 may be adjusted by adjusting the arm member 10 and/or the gripping member 11 (FIG. 4). The medical instrument 14 is then positioned in the “C”-shaped opening 21 of the gripping member 11 (FIG. 5). The location and/or orientation of the medical instrument 14 may be adjusted by adjusting the arm member 10 and/or the gripping member 11 (FIG. 6).

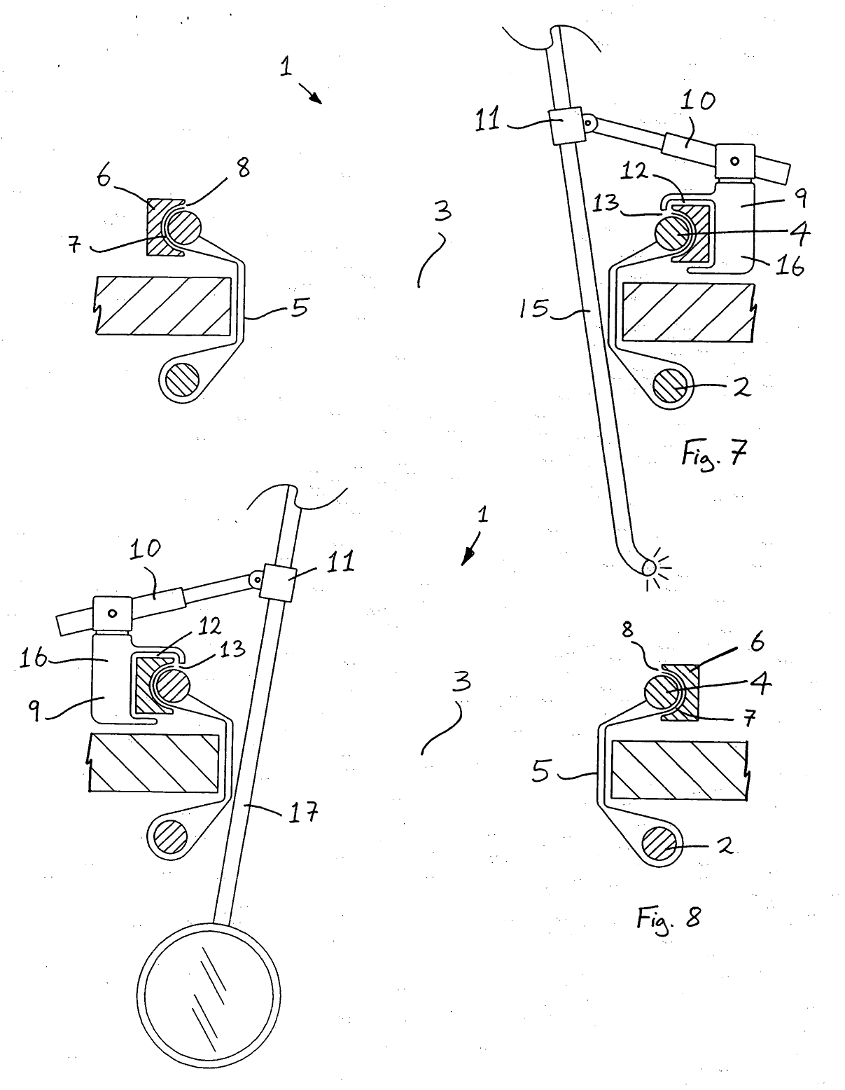

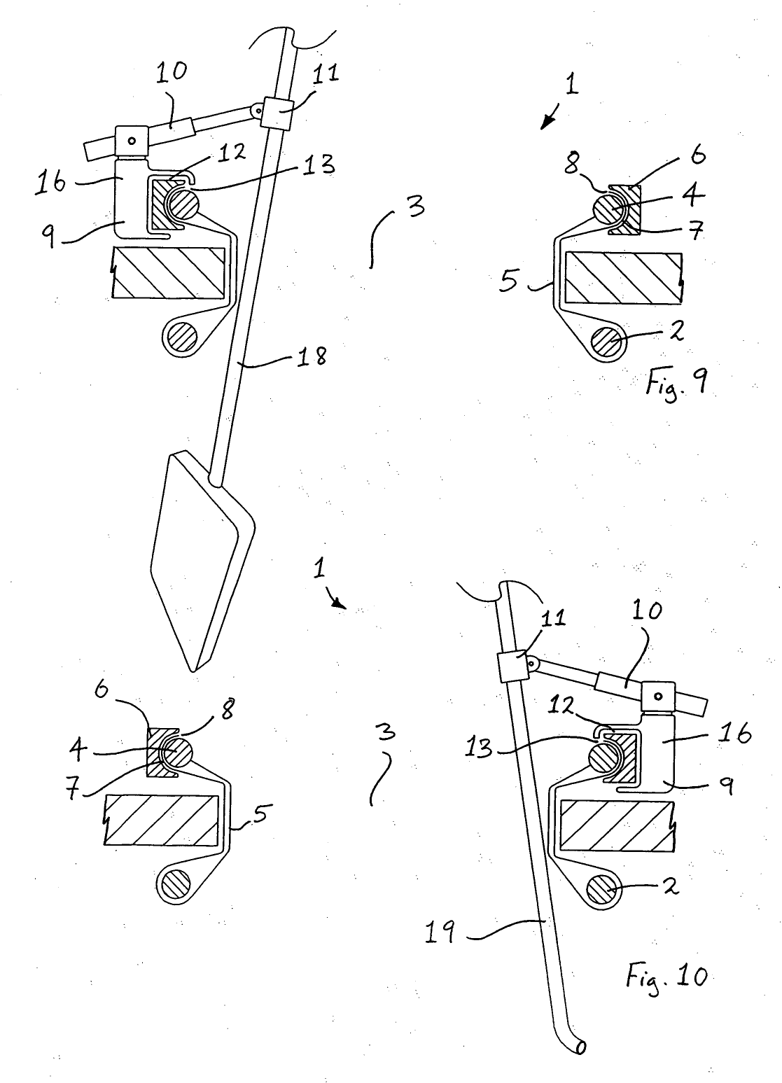

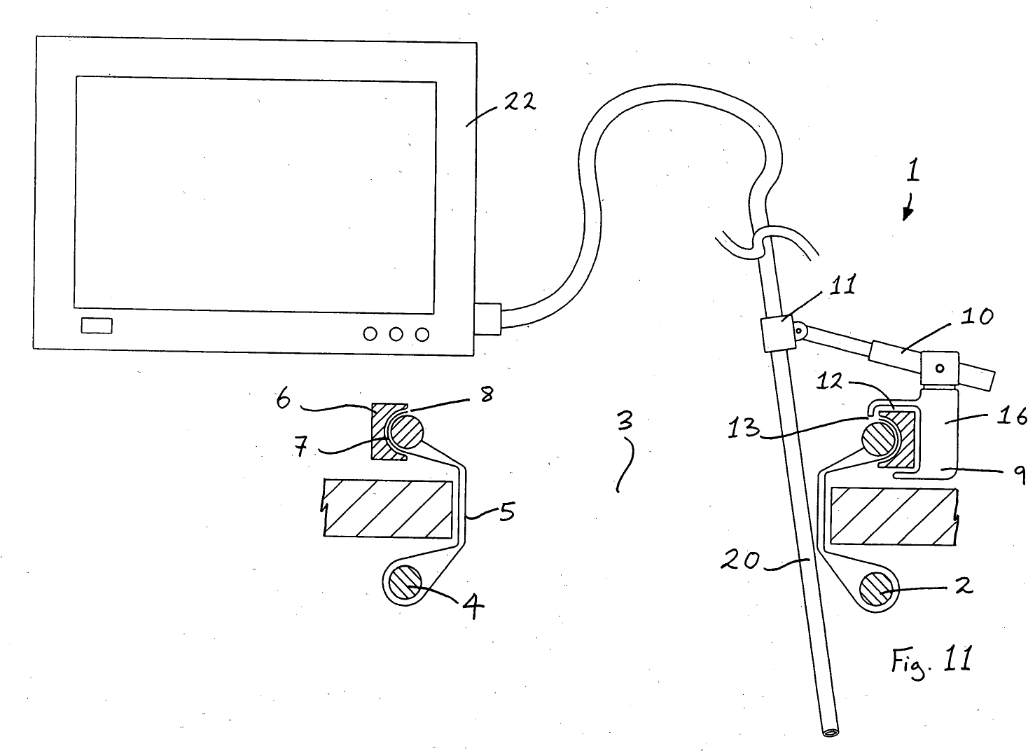

It will be appreciated that the device 1 is suitable for holding a variety of medical instruments in a desired location and/or orientation. For example, a lighting instrument 15 (FIG. 7), and/or a magnifying/mirror instrument 17 (FIG. 8), and/or an internal organ retracting instrument 18 (FIG. 9), and/or a suction/irrigation instrument 19 (FIG. 10), and/or an endoscope instrument 20 operably connected to a monitor 22 (FIG. 11).

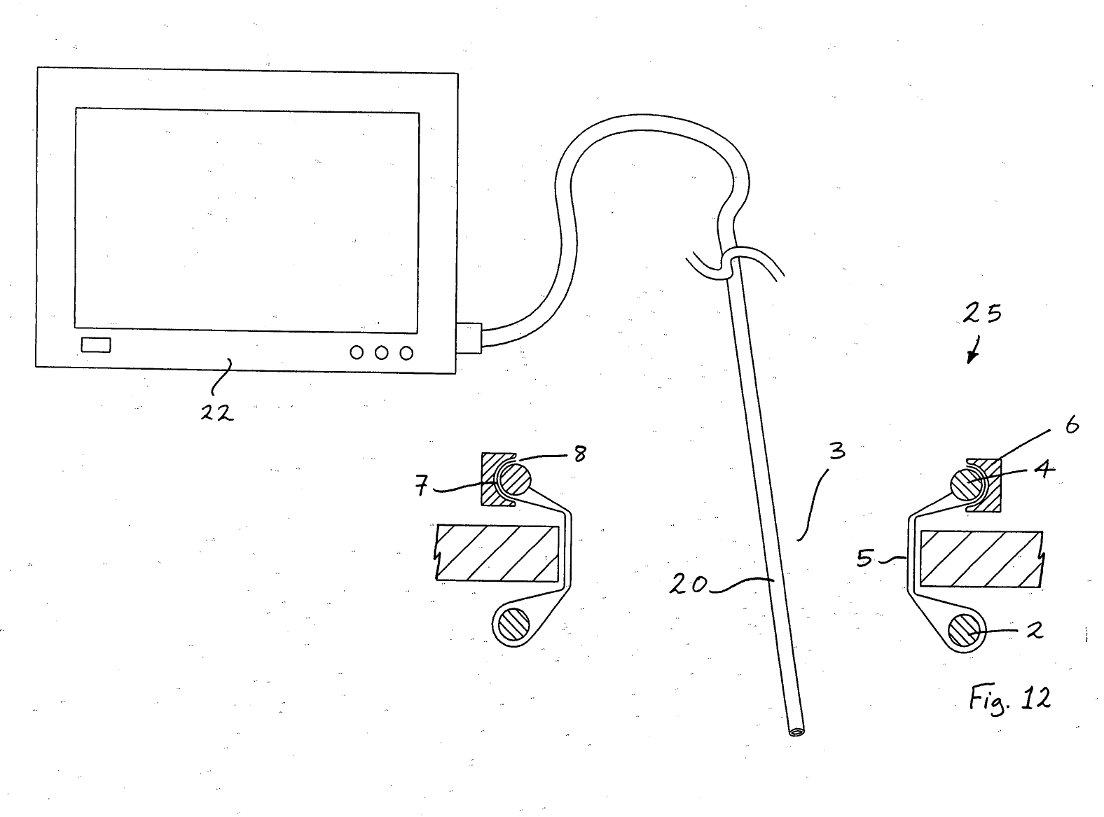

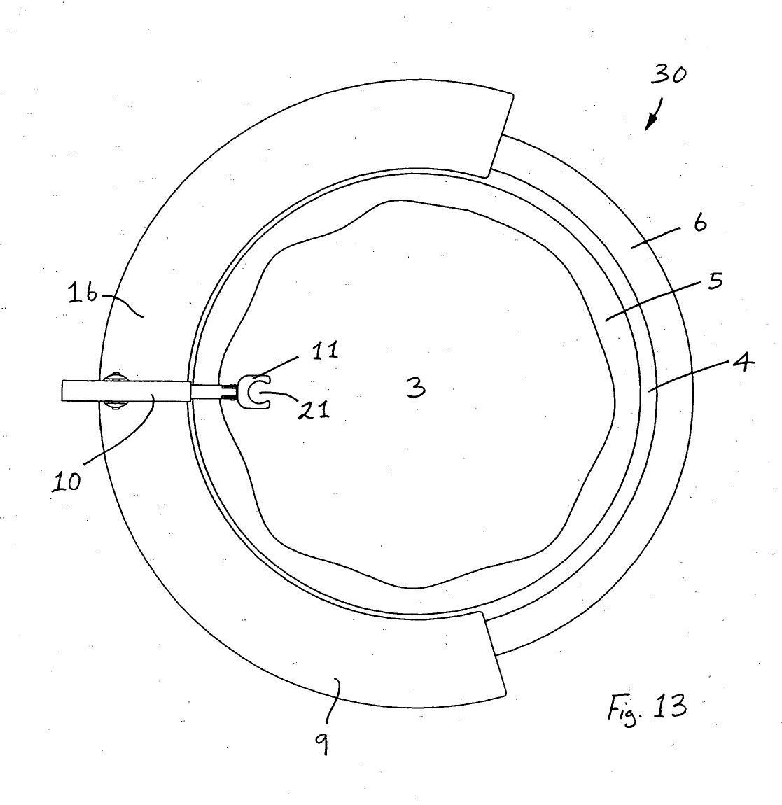

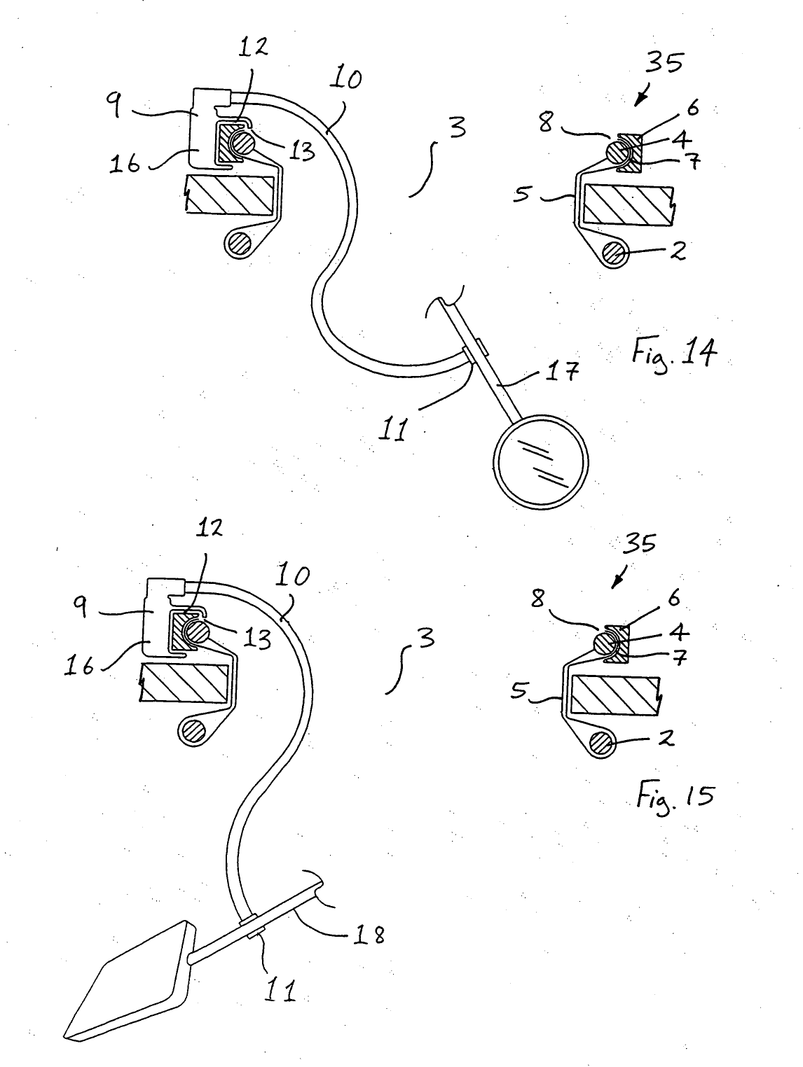

Referring to FIG. 12, there is illustrated another wound retractor device 25, according to the invention, which is similar to the device 1 of FIGS. 1 to 11, and similar elements in FIG. 12 are assigned the same reference numerals.

In this case, the device 25 does not include the medical device 16. Instead the medical instrument, such as the endoscope instrument 20, is inserted through the wound opening 3 and directly held in a desired location and/or orientation by means of a surgeon's hand.

In FIG. 13 there is illustrated another wound retractor device 30 according to the invention, which is similar to the device 1 of FIGS. 1 to 11, and similar elements in FIG. 13 are assigned to the same reference numerals.

In this case, the body portion 9 extends around a relatively large part of the circumference of the platform member 6. In particular, the body portion 9 extends around more than 50% of the circumference of the platform member 6. The greater circumference of this body portion 9 provides enhanced stability to the arm member 10 and the gripping member 11.

FIGS. 14 to 17 illustrate another wound retractor device 35 according to the invention, which is similar to the device 1 of FIGS. 1 to 11, and similar elements in FIGS. 14 to 17 are assigned the same reference numerals.

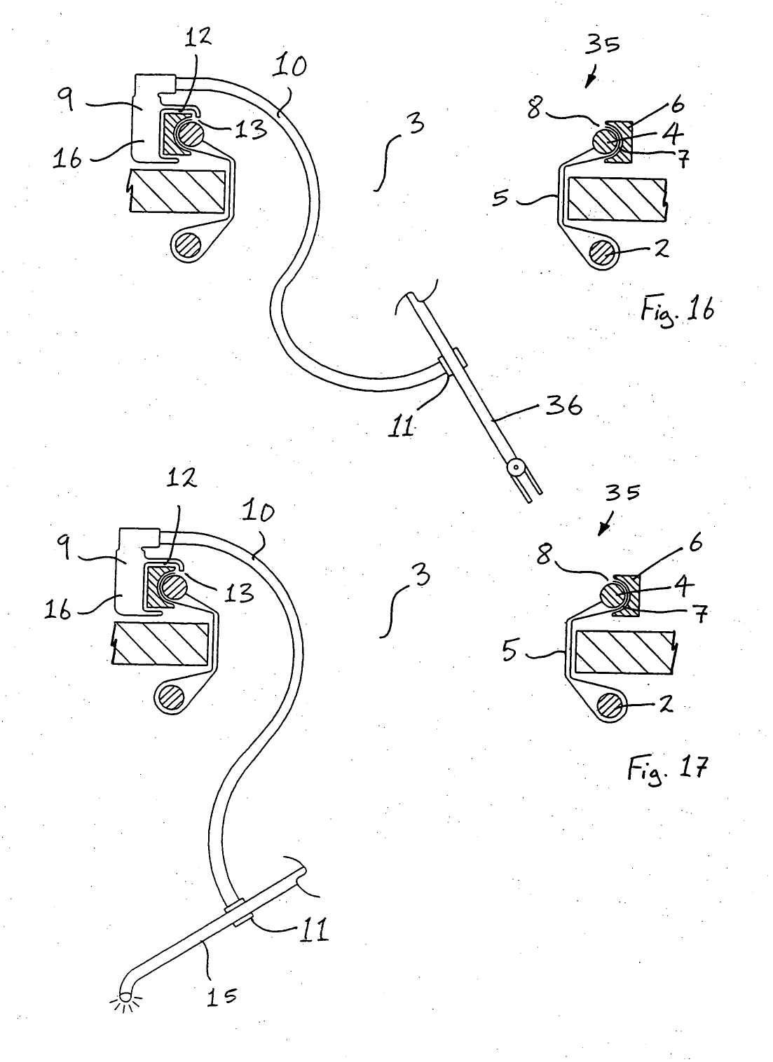

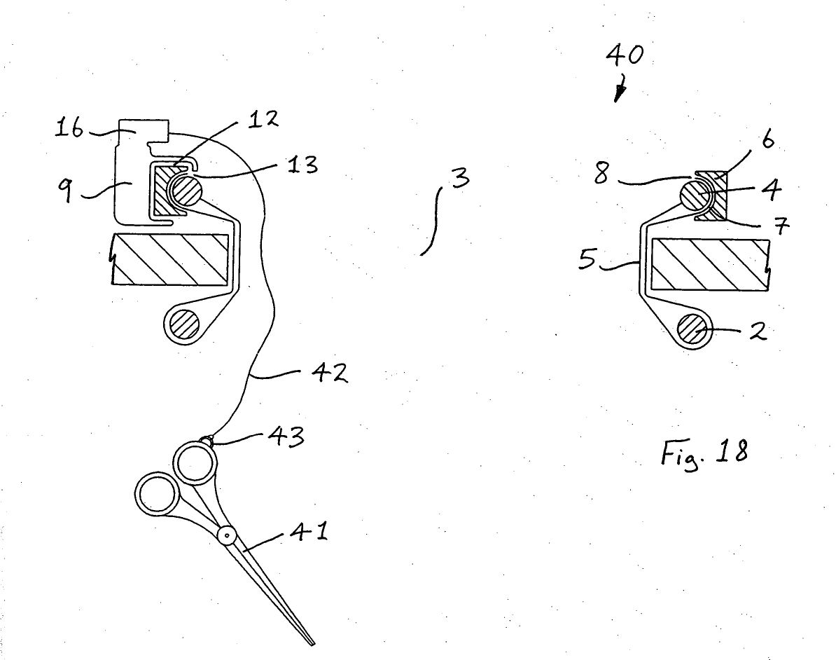

In this case, the arm member 10 is flexible. The arm member 10 is mounted to the body portion 9 in a non-pivoting arrangement, and the gripping member 11 is mounted to the arm member 10 in a non-pivoting arrangement. The flexible nature of the arm member 10 enables the gripping member 11 to be adjusted in three dimensions to adjust the location and/or orientation of the magnifying/mirror instrument 17, as desired (FIG. 14). The device 35 is also suitable for use with a variety of other medical instruments, such as the internal organ retracting instrument 18 (FIG. 15), and/or a gripper/scissors instrument 36 (FIG. 16), and/or the lighting instrument 15 (FIG. 17).

In an alternative embodiment, the arm member 10 may be malleable. In this case the arm member 10 may be arranged/twisted/shaped into a desired configuration; and the malleable arm member 10 will retain this desired configuration.

Referring to FIG. 18 there is illustrated a further wound retractor device 40 according to the invention, which is similar to the device 35 of FIGS. 14 to 17, and similar elements in FIG. 18 are assigned the same reference numerals.

In this case, the medical device 16 comprises a flexible wire 42 to connect the body portion 9 to a medical instrument, in this case a scissors instrument 41. The wire 42 is looped through a hook formation 43 on the scissors instrument 41.

A swab could also be connected to the body portion 9 by means of the wire 42.

It will be appreciated that a cable, or a cord may be employed as an alternative to the wire 42.

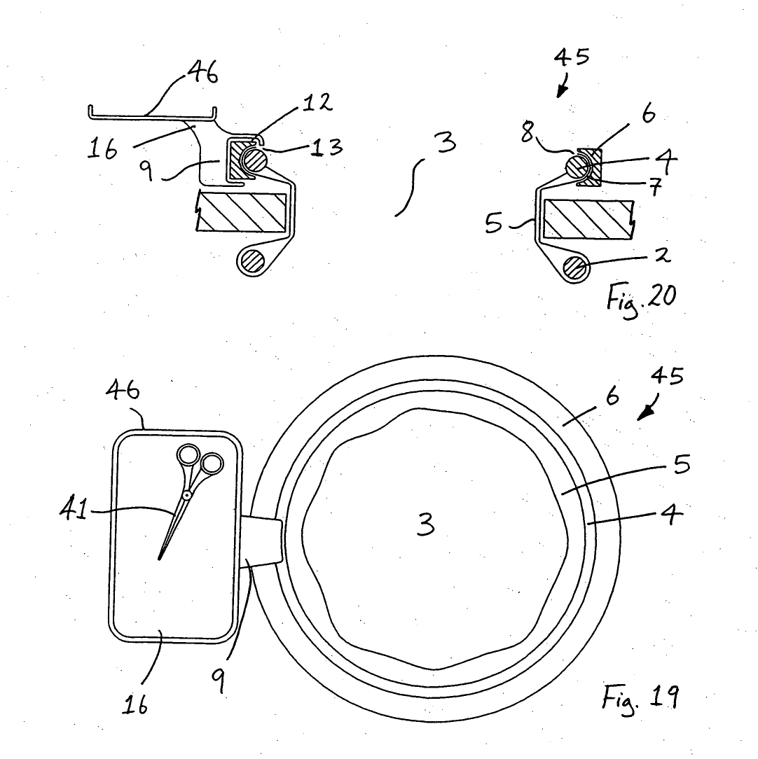

In FIGS. 19 and 20, there is illustrated a further wound retractor device 45 according to the invention, which is similar to the device 1 of FIGS. 1 to 11, and similar elements in FIGS. 19 and 20 are assigned the same reference numerals.

In this case, the medical device 16 comprises a tray 46 to hold a medical instrument, such as the scissors instrument 41, in a desired location and/or orientation.

This tray 46 provides the surgeon with fast and easy access to a scissors instrument 41, and/or swabs, and/or tissue samples and the like.

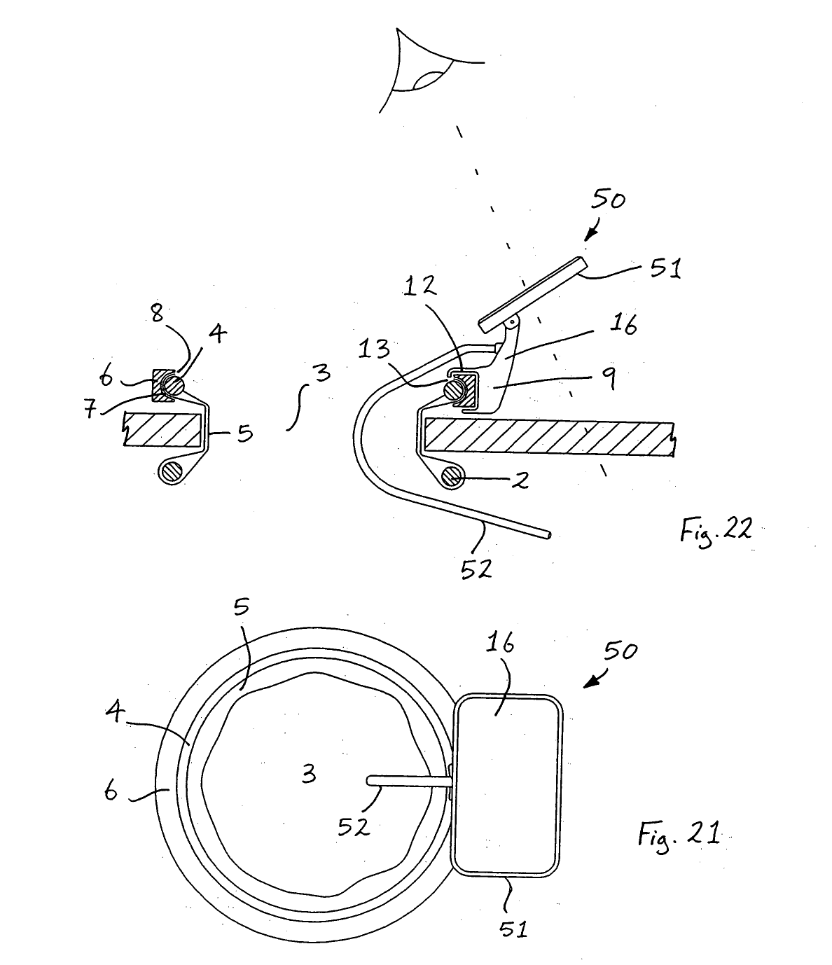

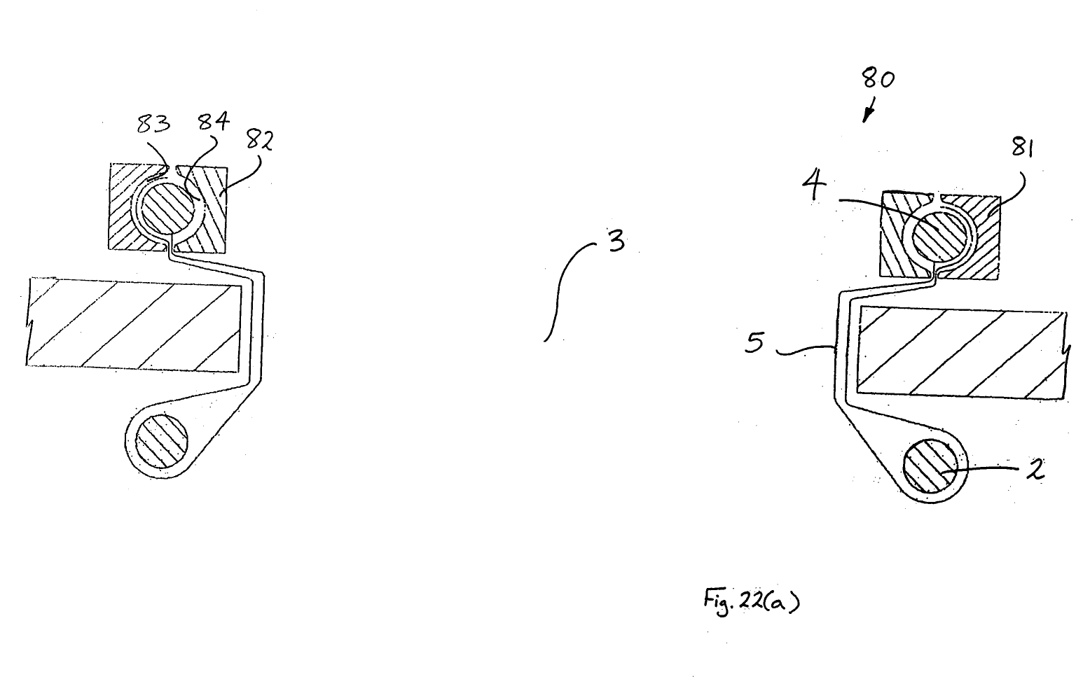

FIGS. 21 and 22 illustrate another wound retractor device 50 according to the invention, which is similar to the device 45 of FIGS. 19 and 20, and similar elements in FIGS. 21 and 22 are assigned the same reference numerals.

In this case, the medical device 16 comprises artificial vision means to view the wound interior. The artificial vision means is provided in the form of a scope 52 for insertion through the wound opening 3, and an LCD monitor 51 to which the scope 52 is operably connected. The location and/or orientation of both the scope 52 and the monitor 51 are adjustable to suit the surgeon.

The artificial vision means provides the surgeon with a closer-zoom view of the site of interest in the wound interior.

In FIG. 22(a) there is illustrated another wound retractor device 80 according to the invention, which is similar to the device 1 of FIGS. 1 to 11, and similar elements in FIG. 22(a) are assigned the same reference numerals.

In this case, the device 80 comprises two rigid, ring-shaped platform members 81, 82. Both platform members 81, 82 are substantially “C”-shaped in cross-section, and comprise female recess 83, 84 into which the proximal ring member 4 is receivable to mount the platform members 81, 82 to the proximal ring member 4 in a snap-fit manner.

As illustrated in FIG. 22(a), the opening of the female recess 84 of the inner platform member 82 faces radially outwardly, and the opening of the female recess 83 of the outer platform member faces radially outwardly.

Either or both of the platform members 81, 82 may be employed to support one or more medical devices in a desired location and/or orientation.

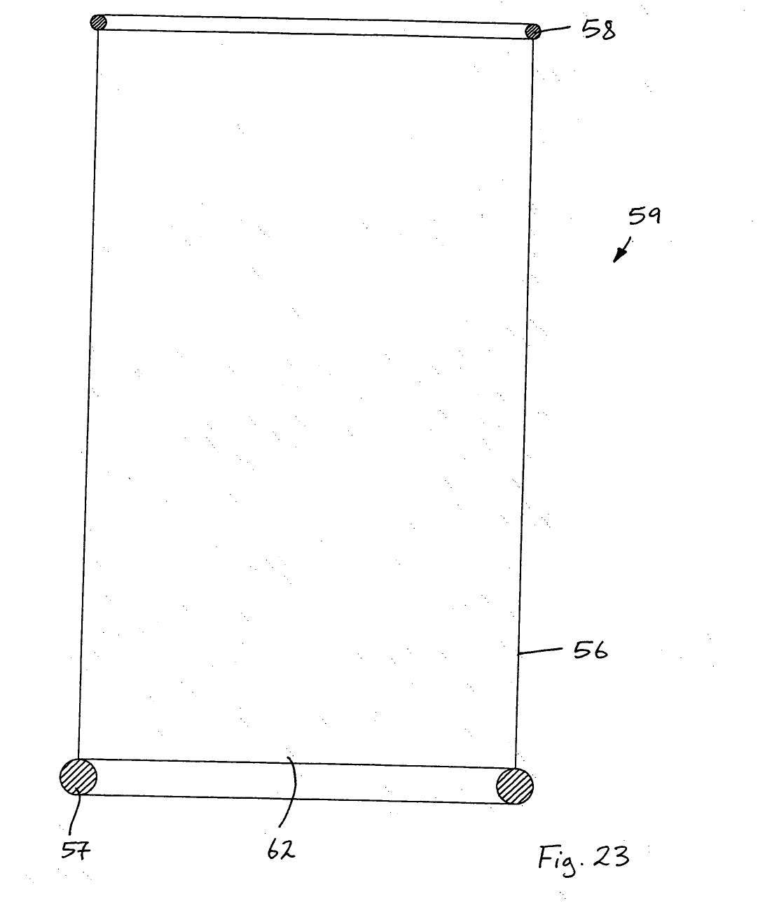

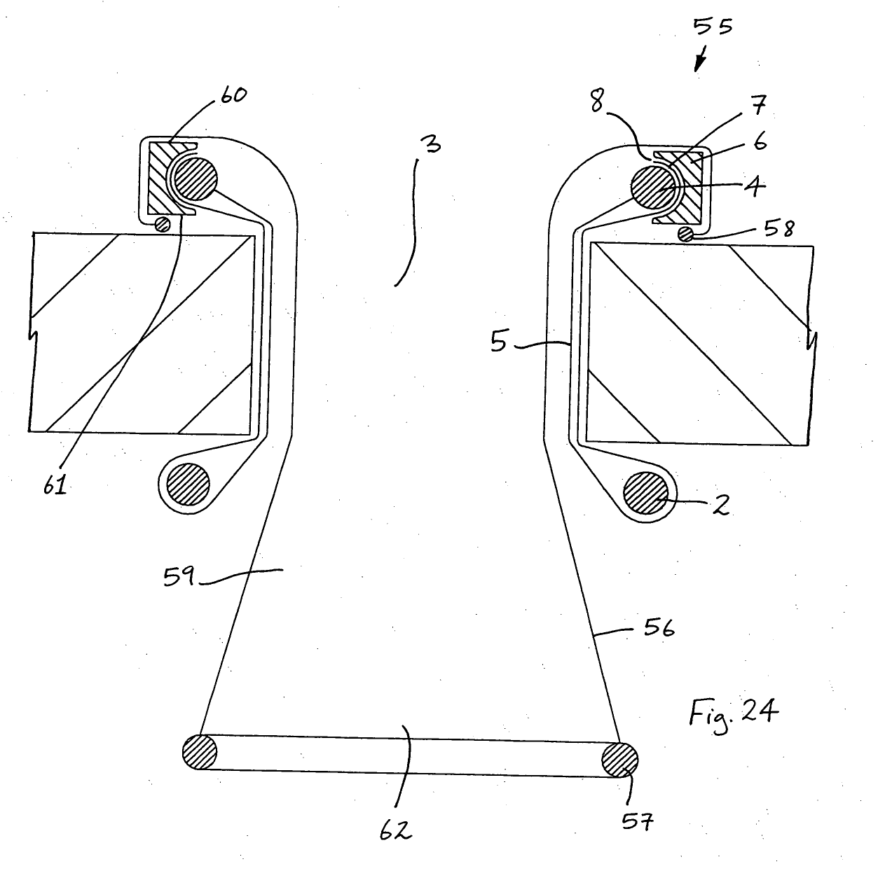

Referring to FIG. 23 and 24, there is illustrated another wound retractor device 55 according to the invention, which is similar to the device 1 of FIGS. 1 to 11, and similar elements in FIGS. 23 and 24 are assigned the same reference numerals.

In this case, the device 55 comprises the distal ring member 2, the proximal ring member 4, the connecting sleeve 5, and the platform member 6. In addition, the device 55 comprises a medical device 59 for retaining internal organs in a desired configuration. The medical device 59 comprises an elasticated proximal anchor ring member 58, a cylindrical film sleeve member 56, and a rigid distal ring member 57 at a distal end of the sleeve member 56 (FIG. 23).

The proximal anchor ring member 58 is configured to be looped around the proximal side 60 of the platform member 6 and anchored at the distal side 61 of the platform member 6 (FIG. 24). In this manner, the medical device 59 is anchored to the platform member 6 to mount the medical device 59 to the platform member 6.

The sleeve member 56 is configured to be extended through the wound opening 3 into the wound interior. In this manner, internal organs adjacent the wound opening 3 may be retained in a desired configuration.

The distal ring member 57 maintains the distal opening 62 of the sleeve member 56 in an open configuration. In this manner obstruction of the working channel through the sleeve member 56 is prevented.

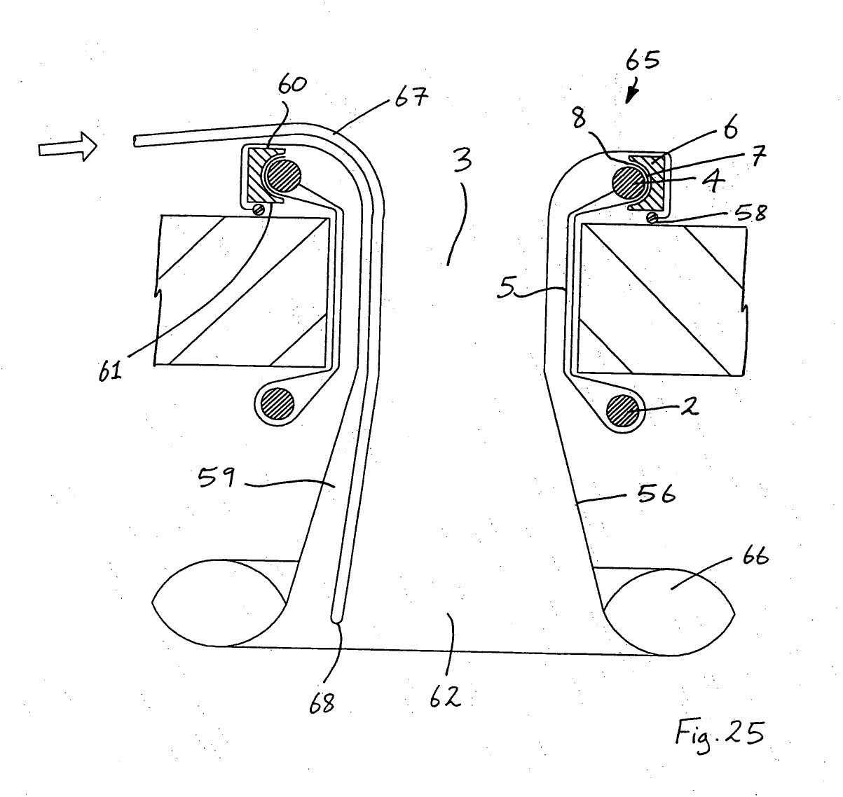

FIG. 25 illustrates another wound retractor device 65 according to the invention, which is similar to the device 55 of FIGS. 23 and 24, and similar elements in FIG. 25 are assigned the same reference numerals.

In this case, the medical device 59 comprises an expandable chamber 66 at the distal end of the sleeve member 56. An inflation/deflation tube 67 extends from externally of the wound opening 3 through the wound opening 3 to an inflation/deflation port 68, where the tube 67 is connected in fluid communication with the expandable chamber 66. The expandable chamber 66 acts to maintain the distal opening 62 of the sleeve member 56 in an open configuration.

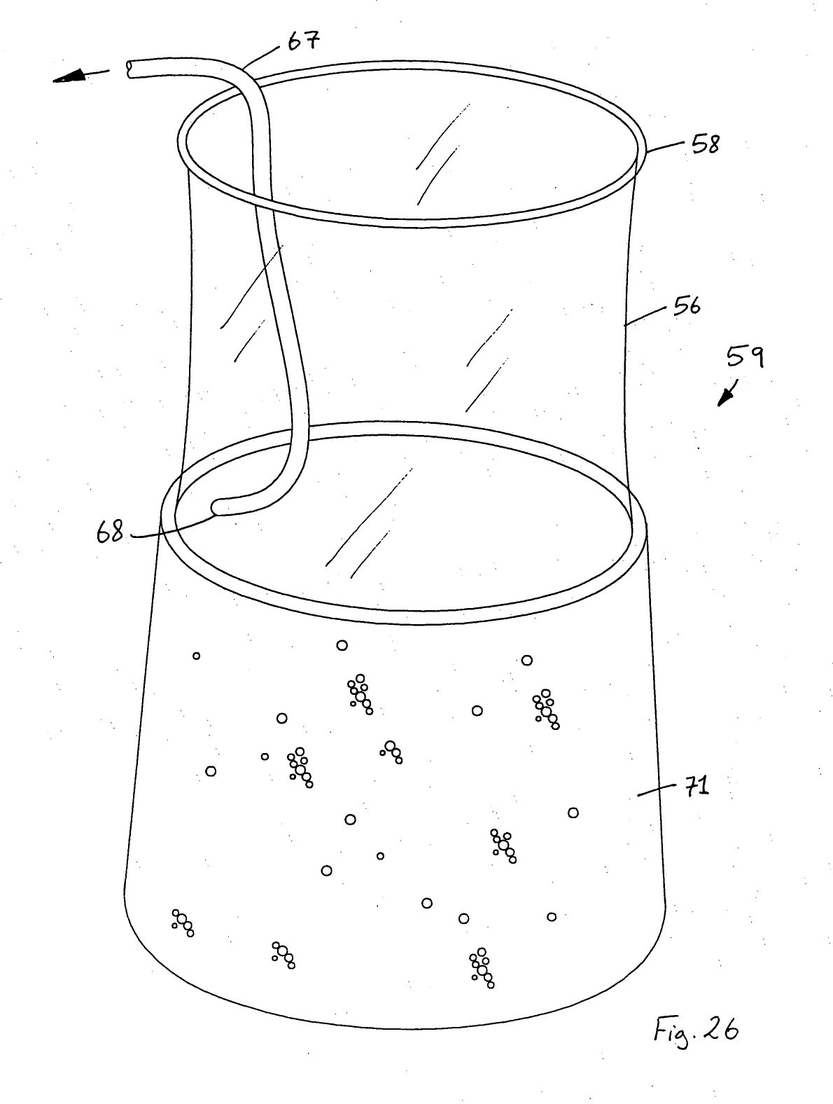

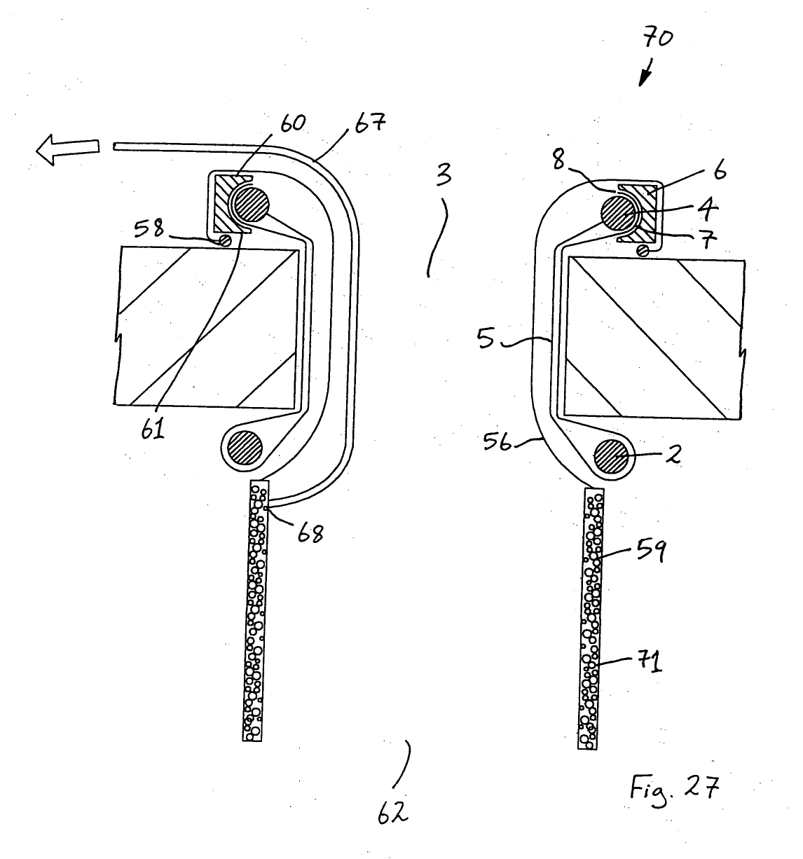

In FIGS. 26 to 28, there is illustrated a further wound retractor device 70 according to the invention, which is similar to the device 65 of FIG. 25, and similar elements in FIGS. 26 to 28 are assigned the same reference numerals.

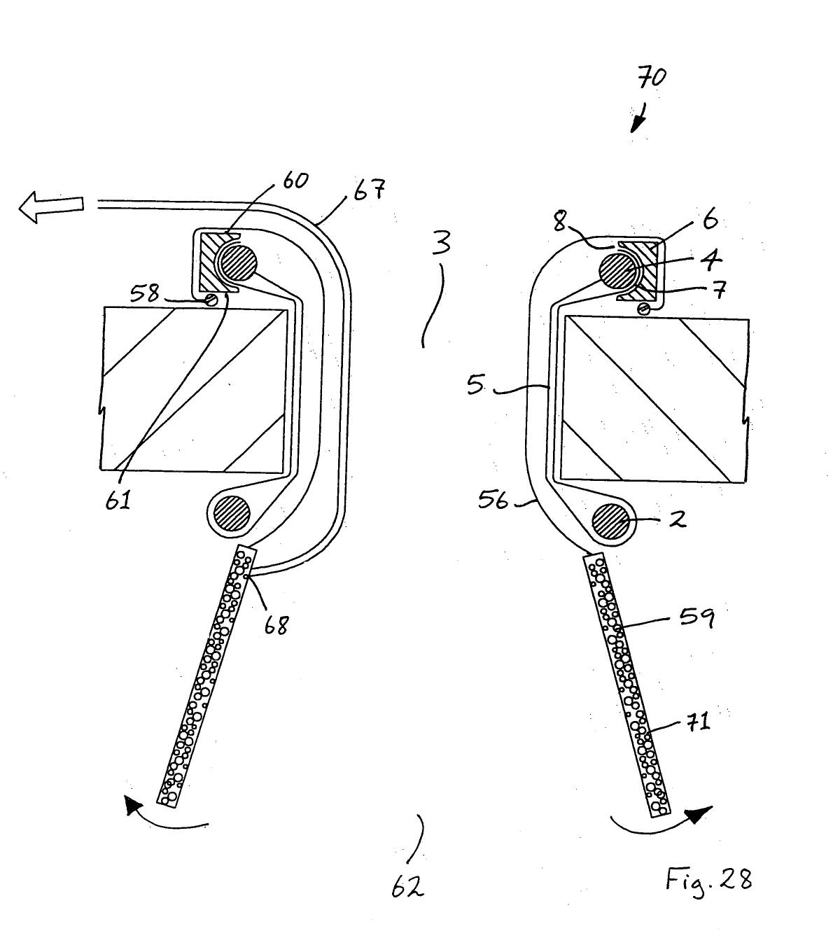

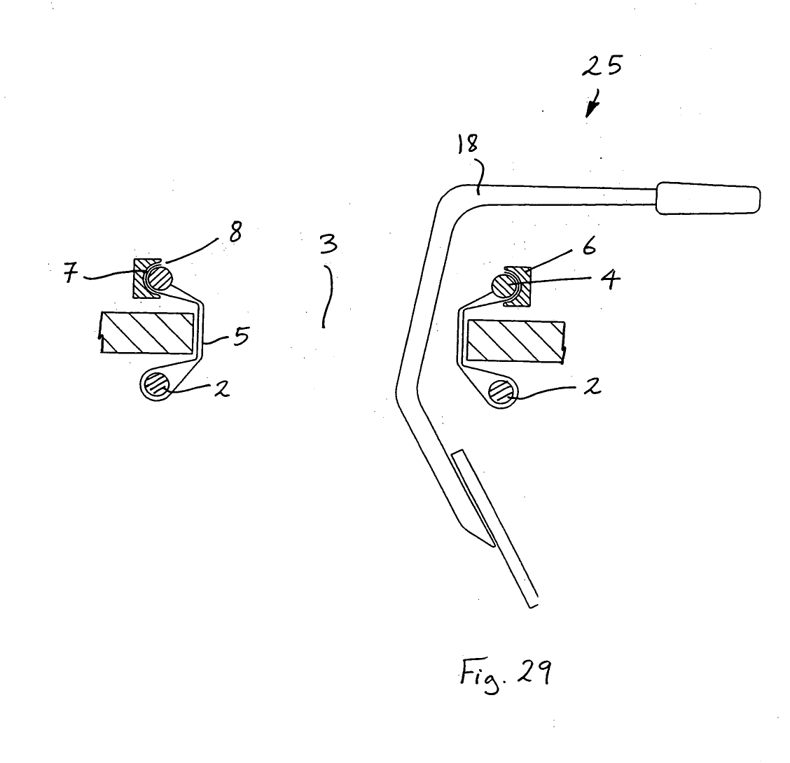

In this case, the medical device 59 comprises an outer envelope 71 having two or more sheets of material arranged in face to face relation in the envelope 71. The envelope 71 is described in further detail in International patent application number PCT/IE00/00128 published under number WO 01/26560, the relevant contents of which are incorporated herein by reference. The envelope 71 has a malleable forming configuration in which the sheets are relatively free to slide over one another in manipulating the envelope 71 to a desired shape (FIG. 27). Upon evacuation of the envelope 71, the envelope 71 has a form retained configuration in which the sheets are restricted from sliding relative to one another (FIG. 28). In this manner internal organs may be retained in a desired configuration.

In FIG. 29, the wound retractor device 25 of FIG. 12 is illustrated with the internal organ retracting instrument 18 extended from externally of the wound opening 3 through the wound opening 3 into the wound interior.

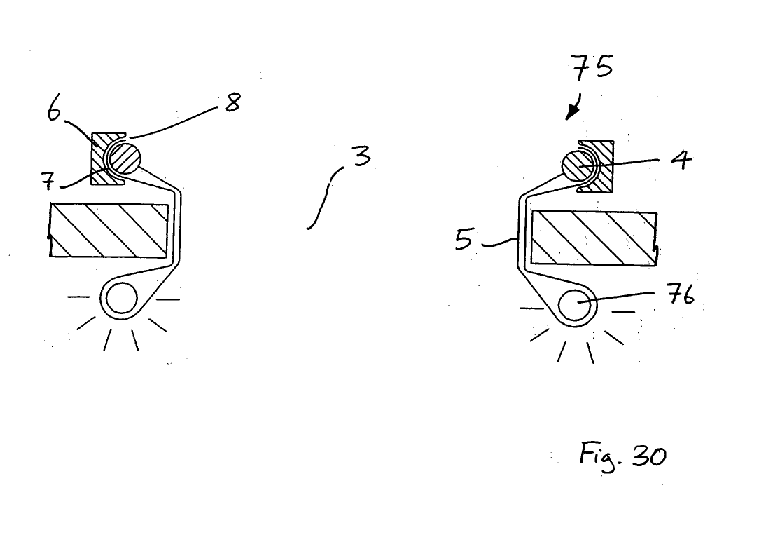

FIG. 30 illustrates another wound retractor device 75 according to the invention, which is similar to the device 25 of FIG. 12, and similar elements in FIG. 30 are assigned the same reference numerals.

In this case, the distal ring member 76 comprises a means to illuminate the wound interior. The illumination means may be provided in any suitable form, such as a luminescent material, and/or an external power source, and/or an internal power source, and/or fibre optics.

It will be appreciated that the support to support the medical device may be provided in a variety of different constructions. For example, in certain cases the support may be provided by the proximal ring member 4 directly.

The invention is not limited to the embodiments hereinbefore described, with references to the accompanying drawings, which may be varied in construction and detail.

1. A wound retractor device comprising:
a distal member for insertion into a wound opening;
a proximal member for location externally of the wound opening;
a connecting member extending between the distal member and the proximal member to retract laterally the sides of the wound opening; and
a support to support one or more medical devices in a desired location and/or orientation.

2. A device as claimed in claim 1 wherein the proximal member comprises the support.

3. A device as claimed in claim 1 wherein the support comprises at least one platform member for location externally of a wound opening.

4. A device as claimed in claim 3 wherein the platform member is mountable to the proximal member.

5. A device as claimed in claim 4 wherein the platform member is mountable to the proximal member in a snap-fit manner.

6. A device as claimed in claim 4 wherein the platform member comprises a female recess into which the proximal member is receivable to mount the platform member to the proximal member.

7. A device as claimed in claim 6 wherein the female recess is substantially “C”-shaped in cross-section.

8. A device as claimed in claim 6 wherein an opening of the female recess faces radially inwardly and/or faces radially outwardly.

9. A device as claimed in claim 1 wherein the proximal member comprises a ring.

10. A device as claimed in claim 3 wherein the platform member comprises a ring.

11. A device as claimed in claim 1 wherein the device comprises a medical device configured to be supported by the support.

12. A device as claimed in claim 11 wherein the medical device is mountable to the support.

13. A device as claimed in claim 12 wherein the medical device is mountable to the support in a snap-fit manner.

14. A device as claimed in claim 12 wherein a body portion of the medical device comprises a female recess into which the support is receivable to mount the medical device to the support.

15. A device as claimed in claim 14 wherein the female recess is substantially “C”-shaped in cross-section.

16. A device as claimed in claim 14 wherein an opening of the female recess faces radially inwardly, and/or faces radially outwardly.

17. A device as claimed in claim 14 wherein the body portion of the medical device extends around a relatively large part of the circumference of the support.

18. A device as claimed in claim 17 wherein the body portion of the medical device extends around more than 50% of the circumference of the support.

19. A device as claimed in claim 14 wherein the body portion of the medical device extends around a relatively small part of the circumference of the support.

20. A device as claimed in claim 19 wherein the body portion of the medical device extends around less than 10% of the circumference of the support.

21. A device as claimed in claim 12 wherein the medical device comprises a proximal anchor member for anchoring the medical device to the support to mount the medical device to the support.

22. A device as claimed in claim 21 wherein the proximal anchor member is configured to be looped around a proximal side of the support and anchored at a distal side of the support.

23. A device as claimed in claim 21 wherein the proximal anchor member comprises a ring.

24. A device as claimed in claim 11 wherein the medical device comprises a holder to hold a medical instrument in a desired location and/or orientation.

25. A device as claimed in claim 24 wherein the holder comprises a gripping member which is substantially “C”-shaped in cross-section.

26. A device as claimed in claim 24 wherein the holder is adjustable to adjust the location and/or orientation of the medical instrument.

27. A device as claimed in claim 24 wherein the holder is connected to a body portion of the medical device by an arm member.

28. A device as claimed in claim 27 wherein the holder is pivotally mounted to the arm member.

29. A device as claimed in claim 27 wherein the arm member is pivotally mounted to the body portion of the medical device.

30. A device as claimed in claim 27 wherein the arm member is extendable.

31. A device as claimed in claim 27 wherein the arm member is rigid.

32. A device as claimed in claim 27 wherein the arm member is flexible.

33. A device as claimed in claim 32 wherein the arm member comprises a wire, or cable, or cord.

34. A device as claimed in claim 24 wherein the holder comprises a tray.

35. A device as claimed in claim 11 wherein the medical device comprises an artificial vision element to view a wound interior.

36. A device as claimed in claim 35 wherein the artificial vision element comprises a scope for insertion through a wound opening.

37. A device as claimed in claim 36 wherein the location and/or orientation of the scope is adjustable.

38. A device as claimed in claim 36 wherein the artificial vision element comprises a monitor to which the scope is operably connected.

39. A device as claimed in claim 38 wherein the location and/or orientation of the monitor is adjustable.

40. A device as claimed in claim 11 wherein the medical device comprises a retainer to retain internal organs in a desired configuration.

41. A device as claimed in claim 40 wherein the retainer comprises a sleeve member configured to be extended through a wound opening into a wound interior.

42. A device as claimed in claim 41 wherein the retainer comprises a maintainer to maintain a distal opening of the sleeve member in an open configuration.

43. A device as claimed in claim 42 wherein the maintainer comprises a distal ring member at a distal end of the sleeve member.

44. A device as claimed in claim 42 wherein the maintainer comprises an expandable chamber at a distal end of the sleeve member.

45. A device as claimed in claim 40 wherein the retainer comprises an outer envelope of pliable material, the envelope having an evacuation port, and at least two sheets of material arranged in face to face relation in the envelope, the retainer having a malleable forming configuration in which the sheets are relatively free to slide over one another in manipulating the retainer to a desired shape, and a form retained configuration in which the sheets are restrained from sliding relative to one another on evacuation of the envelope.

46. A method for retracting a wound opening, the method comprising the steps of:
providing a wound retractor device comprising a distal member, a proximal member, and a connecting member extending between the distal member and the proximal member;
inserting the distal member into the wound opening, and locating the proximal member externally of the wound opening, with the connecting member extending between the distal member and the proximal member to retract laterally the sides of the wound opening; and
supporting one or more medical devices on the wound retractor device in a desired location and/or orientation.

47. A method as claimed in claim 46 wherein the medical device is supported on the proximal member.

48. A method as claimed in claim 46 wherein the wound retractor device comprises at least one platform member located externally of the wound opening, and the medical device is supported on the platform member.

49. A method as claimed in claim 48 wherein the method comprises the step of mounting the platform member to the proximal member.

50. A method as claimed in claim 46 wherein the method comprises the step of mounting a medical device to the wound retractor device.

51. A method as claimed in claim 50 wherein the method comprises the step of anchoring the medical device to the wound retractor device.

52. A method as claimed in claim 50 wherein the method comprises the step of using the medical device to hold a medical instrument in a desired location and/or orientation.

53. A method as claimed in claim 50 wherein the method comprises the step of using the medical device to view a wound interior.

54. A method as claimed in claim 50 wherein the method comprises the step of using the medical device to retain internal organs in a desired configuration.
